(19) United States
(12) Patent Application Publication
(10) Pub. No.: US 2010/0204329 A1
(43) Pub. Date: Aug. 12, 2010

(54) METHOD OF USING AND ESTABLISHING AN ABSORPTION RATE LEVEL AND A NEURON FIRING LEVEL

(76) Inventor: WILLIAM J. McFAUL, JACKSON, NJ (US)

Correspondence Address: KNOBLE, YOSHIDA & DUNLEAVY, EIGHT PENN CENTER, SUITE 1350, 1628 JOHN F KENNEDY BLVD, PHILADELPHIA, PA, 19103, US

(21) Appl. No.: 12/701,076

(22) Filed: Feb. 5, 2010

Related U.S. Application Data
(60) Provisional application No. 61/150,470, filed on Feb. 6, 2009.

(51) Int. Cl. A61K 31/195; A61K 31/197; A61P 43/00

(52) U.S. Cl. 514/562; 514/561

(57) ABSTRACT

A method for modulating the homeostatic relationship between various amino acid neurotransmitters in the body. The method includes modulating the rate of neuron firing and modulating the rate of cellular absorption.

This Application claims the benefit of U.S. Provisional Application Ser. No. 61/150,470, filed Feb. 6, 2009, the contents of which are incorporated herein by reference.

### BACKGROUND OF THE INVENTION

1. Field of the Invention

The present invention relates to the field of amino acid neurotransmitters. In particular the present invention relates to the identification and regulation of substances that control the homeostatic relationship of pairs of amino acid neurotransmitters.

2. Description of the Related Technology

All forms of life are supported by a variety of substances; including but not limited to hormones, proteins, peptides, amino acids, minerals (ions), vitamers (chemicals or compounds that function as vitamins) and bacteria, minerals, genes, enzymes, lipids (i.e. fatty acids), carbohydrates, amino acids, ions and gasotransmitters. These substances create the homeostasis essential to maintain life for humans as well as plants and animals.

Current scientific findings have not identified that the amino acid neurotransmitters—glutamate and glycine as well as aspartate and gamma aminobutyric acid (GABA) are pairs that regulate the body's rate of cellular absorption and the firing rate of neurons and signaling mechanisms throughout the body. In addition, medical science has failed to recognize the fact that the homeostatic relationships between these amino acid neurotransmitters are regulated by other substances that can assist in placing the amino acid neurotransmitter back in homeostatic equilibrium.

Therefore, there is a need in the field to identify and regulate the homeostatic relationships between the amino acid neurotransmitters that are important to the body's functioning.

### SUMMARY OF THE INVENTION

An object of the invention is determining whether or not glutamate and glycine are within homeostatic balance.

Another object of the invention determining whether or not aspartate and GABA are within homeostatic balance.

Yet another object of the invention is administering cysteine when glutamate and glycine are not within homeostatic balance.

Still yet another object of the invention is administering glutamate that converts to the enzyme glutamate decarboxylase (GAD) or administering GAD itself when aspartate and GABA are not within homeostatic balance.

An aspect of the present invention is a method for establishing homeostasis for cellular absorption comprising: measuring a level of glutamate in an individual; measuring a level of glycine in the individual; calculating a ratio of the level of glutamate to the level of glycine; and administering cysteine to the individual when the ratio is not within a predetermined range.

Still yet another aspect of the present invention is a method for obtaining homeostasis in an individual comprising: measuring a level of a first substance in an individual; measuring a level of second substance in an individual; calculating a ratio of the level of the first substance to the level of the second substance; and administering a third substance to the individual when the ratio is not within a predetermined range.

Another aspect of the invention may be a method for determining the level of cellular absorption in an individual comprising: measuring a level of glutamate in an individual; measuring a level of glycine in the individual; measuring a level of cysteine in the individual; and determining whether or not homeostasis has been obtained.

These and various other advantages and features of novelty that characterize the invention are pointed out with particularity in the claims annexed hereto and forming a part hereof. However, for a better understanding of the invention, its advantages, and the objects obtained by its use, reference should be made to the drawings which form a further part hereof, and to the accompanying descriptive matter, in which there is illustrated and described a preferred embodiment of the invention.

### BRIEF DESCRIPTION OF THE DRAWINGS

FIG. 1 shows a diagram illustrating the homeostatic relationship.

FIG. 2 shows a diagram illustrating the homeostatic relationship of glutamate, glycine and cysteine.

FIG. 3 is a flow chart of the method for establishing homeostasis of glutamate, glycine.

FIG. 4 shows a diagram illustrating the homeostatic relationship of aspartate, GABA and glutamate or GAD.

FIG. 5 is a flow chart of the method for establishing homeostasis of aspartate and GABA.

### DETAILED DESCRIPTION OF THE PREFERRED EMBODIMENT(S)

All forms of life are supported by modulated pairs of substances; including but not limited to hormones, proteins, peptides, amino acids, minerals (ions), vitamers (chemicals or compounds that function as vitamins) and bacteria minerals, genes, enzymes, lipids (i.e. fatty acids), carbohydrates, amino acids, ions and gasotransmitters. These substances create the homeostasis essential to maintain life for humans as well as plants and animals.

Several terms are used herein and are defined as follows:

The term “modulator” means an agent, substance or mechanism that facilitates the maintenance of homeostasis between pairs. “Pairs” may be hormones, minerals, proteins, amino acids, bacteria, virus, and natural processes. The agent can decrease intensity of stimulatory processes or substances, or increase intensity of compensatory ones.

The term “catalyst” means the driver which initiates modulation to take place. Such catalysts can include enzymes, hormones, RNA, and countless signaling mechanisms.

The term “substance” refers to compounds, elements, ions and molecules and combinations thereof.

The term “neuron” means the signaling molecules or signaling mechanisms.

The term “Homeostatic Relationship (Homeostasis)” means the balance or equilibrium between two substances. The relationship does not have to be 50%-50% but can vary depending upon the two substances in question.

The invention addresses the diagnostic analysis of the corollary relationships between amino acids that provide and maintain homeostasis; i.e. pairs which include substances that are stimulatory/excitatory (e.g. glutamate and aspartate) and those that are compensatory/inhibitory (e.g. glycine and gamma aminobutyric acid). While medical science has identified the nature of these amino acids (e.g. excitatory or inhibitory), they have not identified their specific roles, the relationships between them or the existence of substances that modulate the relationships between them.

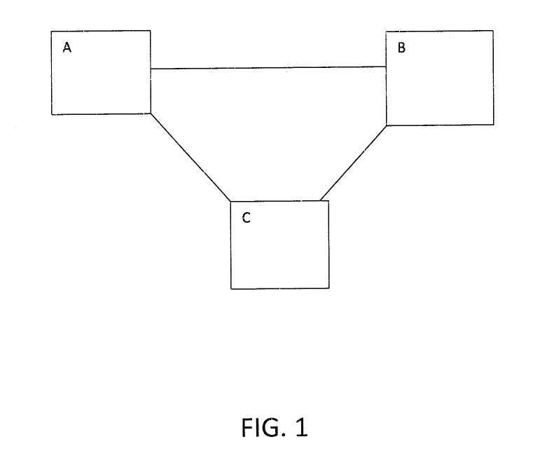

The concept of modulation in relation to homeostatic pairs is illustrated in FIG. 1. This example illustrates a relationship between two substances, A and B, and the existence of a third substance C that serves to support A or B in order to attempt to maintain an adequate level of homeostasis (balance) between the two substances.

Current methodologies do not account for processes associated with fluctuations of or imbalances between cellular absorption rates and the rates of firing of neurons. Medical science knows that minerals and certain amino acids bind to various types of cells. It is assumed that the binding process occurs as the mechanism by which all cells absorb all substances (also referred to as intake or uptake). Instead, the rate of cellular absorption is determined by the homeostatic relationship between glutamate and glycine.

The natural mechanism that facilitates the homeostatic relationship for maintaining adequate cellular absorption is the pairing of glutamate and glycine and utilization of cysteine. In instances where an imbalance occurs that has the potential of disrupting homeostasis, cysteine serves to modulate the imbalance.

A low or slow rate of cellular absorption will retard the body's ability to process substances, other than neurons, to maintain homeostatic relations or other systems and processes throughout the body. Conversely, a greater or higher rate of cellular absorption will disrupt homeostatic relationships throughout the body by accelerating systems and processes. Examples of an accelerated rate of absorption are allergic reactions resulting from substances that might not impact individuals with lower rates of cellular absorption and cancers that may result when cells absorb greater levels of toxins.

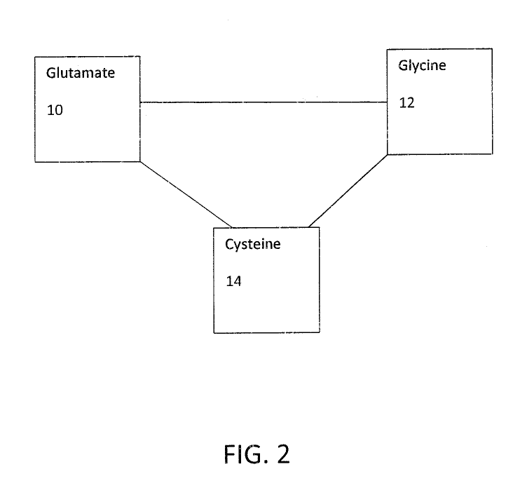

For example, homeostasis for cellular absorption rates is determined by the pairing of glutamate 10, which controls the increase in absorption rate with glycine 12, which decreases the absorption rate. The amino acid cysteine 14 serves as the modulator for the process. Application of cysteine 14 increases the level of glutamate 10 within the body. This relationship is diagrammatically illustrated in FIG. 2.

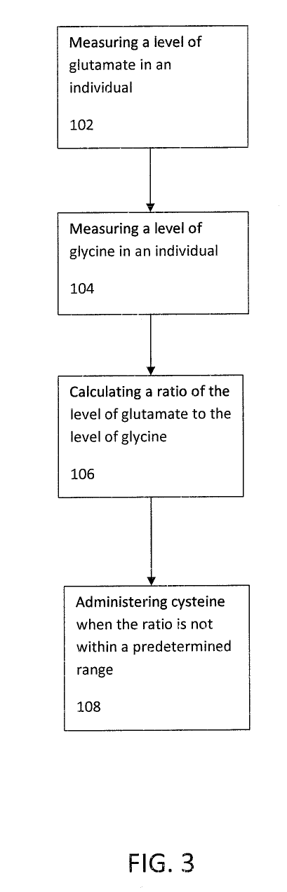

FIG. 3 shows the method of returning the levels of glutamate and glycine to homeostatic balance. In step 102 the level of glutamate 10 is measured in an individual. The level of glutamate 10 may be measured by a variety of diagnostic tests that include but are not limited to the analysis of diagnostic imaging that indicates activity and the analysis of various fluids and excretions within and produced by the body. Examples of these fluids and excretions include: blood and its components; urine; fecal matter; collagen; chyle; interstitial fluid (tissue fluid); lymph; extracellular fluid; amniotic fluid; sweat; tears; saliva; mucus; phlegm; hair; fingernails; bone marrow. The measured level of glutamate 10 in the body is expressed in various units depending upon the test employed in order to measure the level of glutamate 10. This level can be expressed by the variable X. There is a preferred range in which X may fall that varies depending upon the type of test that is used in order to measure the level of glutamate 10. In an embodiment of the present invention a standard type of test is used, such as blood test.

In step 104 the level of glycine 12 in an individual is measured. This level can be measured using one of the various processes listed above. The measured level of glycine 12 is preferably expressed in the same units of measurement as that used in the measurement of glutamate 10 so as to more readily compare the levels with respect to each other. This level can be expressed by the variable Y. There is a preferred range in which Y may fall that varies depending upon the type of test that is used in order to measure the level of glycine 12. In an embodiment of the present invention a standard type of test is used, such as a blood test.

In step 106 the level of glutamate 10 that has been measured, X, is compared to the level of glycine 12, Y, in order to obtain ratio. This ratio, X/Y, can be expressed by the variable Z. The variable Z preferably falls within a predetermined range which expresses whether or not glutamate 10 and glycine 12 are within homeostatic balance.

In step 108, if it is determined that the ratio Z is not within the predetermined range, then cysteine 14 is administered to the individual in order to restore the glutamate 10 and glycine 12 levels to a preferred range. Administration of cysteine 14 may be orally administered, e.g., pills, solutions, lozenges, etc.; intravenously administered solutions; modification of dietary supplements and/or other standard methods of delivery. After a period of time after administration, the levels of each of the substances may be retested in order to ascertain whether or not the administration was effective. Additionally, the level of cysteine may be measured in order to determine if more should be administered.

An example of this is provided below using hypothetical numbers so as to make understanding of the process easier. Measured results may further be normalized to a set scale in order to make comparison and calculation easier provided the all values are expressed in the same units. Actual numbers from a test such as a blood test are typically expressed in units of mmol/L.

Measuring glutamate 10 via a blood test results in a number for X that results in a number 3, from within a range of 1-10. Measuring glycine 12 via a blood test results in a number for Y of 5 from within a range of 1-10. The number Z that results from this is 0.6. A preferred range for Z is between 0.8 and 1.2. By administering cysteine 14 to the individual the level of glutamate 10 is increased so that on a subsequent test the level of glutamate 10 results in the number 4 for X, while the number for Y remains at 5. This results in a Z of 0.8, which falls within the predetermined range and indicates that glutamate 10 and glycine 12 are in homeostatic balance and no further application of modulator cysteine 14 is required.

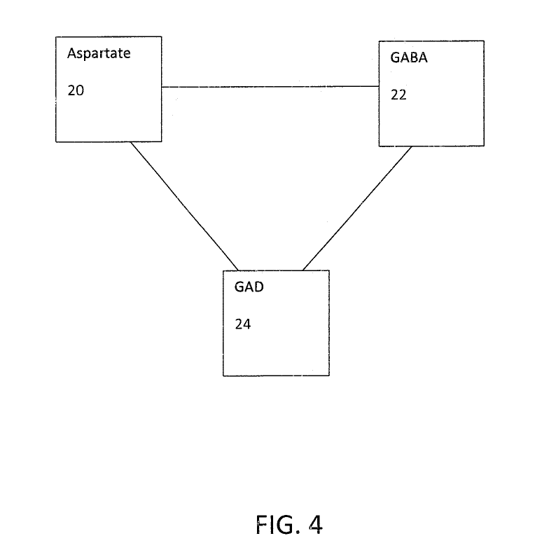

Another example of a modulated homeostatic relationship is that certain cells emit neurons while other cells receive and convert these neurons into various activities. The rate of neuron emission is determined by a homeostatic relationship between aspartate 20 and gamma aminobutyric acid (GABA) 22 and is modulated by glutamate decarboxylase (GAD) 24 This relationship is shown in FIG. 4. As glutamate levels become excessive, the enzyme GAD is synthesized from the glutamate and it acts as the catalyst that converts the excess glutamate into GABA in order to slow the firing rate (between aspartate 20—faster and GABA 22—slower). Administering glutamate would result in the synthesis of GAD or GAD itself could be administered to increase GABA in order to slow neuron firing rates. Alternatively, administering GABA may also reduce excessive firing rates.

Aspartate 20 governs the increase in the firing rate and GABA 22 governs the decrease in firing rate. GAD 24 is the catalyst used to convert glutamate into GABA 22 in order to enable GABA 22 to modulate, i.e. reduce, excessive firing rate in order to maintain a homeostatic relationship between the rate of neural firing of some cells and the ability of other cells to absorb the neurons.

If the rate of neural firing increases, levels of glutamate 24 should increase in order create the enzyme GAD to insure the rate of absorption of the neurons will be maintained in order to sustain the necessary homeostatic relationship.

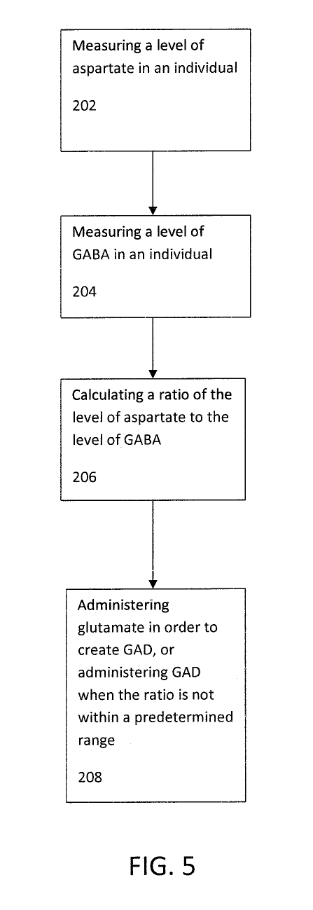

FIG. 5 shows the method of returning the levels of aspartate 20 and GABA 22 to homeostatic balance. In step 202 the level of aspartate 20 is measured in an individual. The level of aspartate 20 may be measured by a variety of diagnostic tests that include but are not limited to the analysis of diagnostic imaging that indicates activity and the analysis of various fluids and excretions within and produced by the body. Examples of these fluids and excretions include: blood and its components; urine; fecal matter; collagen; chyle; interstitial fluid (tissue fluid); lymph; extracellular fluid; amniotic fluid; sweat; tears; saliva; mucus; phlegm; hair; fingernails; bone marrow. The measured level of aspartate 20 in the body is expressed in various units depending upon the test employed in order to measure the level of the aspartate 20. This level can be expressed by the variable X. There is a preferred range in which X may fall that varies depending upon the type of test that is used in order to measure the level of aspartate 20. In an embodiment of the present invention a standard type of test is used, such as a blood test.

In step 204 the level of GABA 22 in an individual is measured. This level can be measured using one of the various processes listed above. The measured level of GABA 22 is preferably expressed in the same units of measurement as that used in the measurement of aspartate 20 so as to more readily compare the levels with respect to each other. This level can be expressed by the variable Y. There is a preferred range in which Y may fall that varies depending upon the type of test that is used in order to measure the level of GABA 22. In an embodiment of the present invention a standard type of test is used, such as a blood test.

In step 206 the level of aspartate 20 that has been measured, X, is compared to the level of GABA 22, Y, in order to obtain ratio. This ratio, X/Y, can be expressed by the variable Z. The variable Z preferably falls within a predetermined range which expresses whether or not aspartate 20 and GABA 22 are within homeostatic balance.

In step 208, if it is determined that the ratio Z is not within the predetermined range, then, GABA 22, glutamate that is converted to GABA 22 by GAD 24 or GAD 24 itself is administered to the individual when the ratio is not within a predetermined range. The substance is administered to the individual in order to restore the aspartate 20 and GABA 22 levels to a preferred range. Administration may be accomplished by provision of orally administered substances e.g. pills, solutions, lozenges, etc.; intravenously administered solutions; modification of dietary supplements and/or other standard methods of delivery. After a period of time, the levels of each of the substances may be retested in order to ascertain whether or not the administration was effective. Additionally, the level of GAD or glutamate may be measured in order to determine if more should be administered.

An example of this is provided below using hypothetical numbers so as to make understanding of the process easier. Measured results may further be normalized to a set scale in order to make comparison and calculation easier provided the all values are expressed in the same units. Actual numbers from a test such as a blood test are typically expressed in units of mmol/L.

Measuring aspartate 20 via a blood test results in a number for X that results in a number 5, from within a range of 1-10. Measuring GABA 22 via a blood test results in a number for Y of 3 from within a range of 1-10. The number Z that results from this is 1.6. A preferred range for Z is between 0.8 and 1.20. By administering glutamate or GAD 24 to the individual the level of GABA 22 is increased so that on a subsequent test the level of GABA results in the number 4.5 for Y, while the number for X remains at 5. This results in a Z of 1.1, which falls within the predetermined range and indicates that aspartate 20 and GABA 22 are in homeostatic balance and no further application of modulator glutamate or GAD 24 is required.

In addition to the analysis of levels of amino acids, including amino acid neurotransmitters, various existing and yet to be developed diagnostic processes that measure outcomes involving these substances can be utilized. Examples of such methodologies include but are not limited to EEG, PET scans, use of MEG machines, SPECT analysis, functional and diffusion MRI technologies as well as other iterations thereof (fMRI and dMRI respectively), CT scans, and ultrasound.

The correlation between base values expressed when the range of homeostasis is determined and an individual's test results can be used as a means of assessing levels or disruptions between pairs or the existence of increases in the levels of modulators in order to maintain homeostasis. It is also contemplated that methods of diagnostic analysis may also be used to detect the presence of catalysts as markers for the existence of a disruption within a modulated pair that the body is in the process of remediating. Detection of such catalysts may be included as a factor in the process of analyzing the correlation of substances within modulated pairs.

The present invention provides the biological foundation that will enable the monitoring of relationships of modulated pairs of amino acid neurotransmitters as they relate to medical care; including wellness, prevention and treatment pertaining to the mind and body.

The following are examples of the benefits of assessing and monitoring the firing rate of neurons and the rate of cellular absorption.

(1) Medications and herbal preparations can be ineffective or cause serious consequences if dosage does not correspond properly to absorption. Creating a baseline assessment of an individual's homeostatic absorption rate can provide a foundation from which dosing can be tailored to meet specific requirements.

(2) Assessment of homeostatic imbalances of cellular absorption rates, and neural firing rates and the relationship between firing rates and absorption rates can enable therapies to correct these disruptions instead of waiting to the point where mental or physical consequences will require expensive or sophisticated treatment of ailments or diseases that are the outcomes.

(3) Rates of activity and absorption will dictate the rate of use or dissipation of nutrients essential to provide energy to maintain bodily processes and functions; including but not limited to the creation and maintenance of the body's defenses (i.e. immune system).

(4) Assessment of the neural firing rates in the brain and the ability to absorb these neurons as a means of determining potential root causes of neuro-developmental or neurodegenerative diseases and disorders as well as the efficacy of treatment regimens. Such diseases and disorders can include but should not be limited to Parkinson's, schizophrenia, Asperger's, attention deficit hyperactivity disorder, obsessive compulsive disorder, depression and Alzheimer's.

(5) Assessment of the impact of various substances on the firing rate of neurons and signaling mechanisms and their impact on the body's rates of activities. This aspect of the invention can enable evaluation of the impact of disruptions from imbalances in amino acid neurotransmitters that may result in signaling mechanisms that may cause hormone, organ or gland imbalances. Assessment of these processes can prevent diseases or enable treatment regimens for diabetes, obesity resulting from disruptions for when to store fat, thyroid imbalances as well as numerous other disease entities.

(6) Assessment of a cell's ability to absorb hormones, immune defenses and other essential substances in order to maintain homeostasis throughout the body

(7) Provide a biologically verifiable means to gauge the efficacy between branded drugs, genetic equivalents, therapeutic equivalents, herbal remedies and other substances and treatments to offer effective means of attaining or maintaining homeostatic levels of cellular absorption, neural firing rates and the relationship between these two processes.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

What is claimed is:

1. A method for establishing homeostasis for cellular absorption comprising:
measuring a level of glutamate in an individual;
measuring a level of glycine in the individual;
calculating a ratio of the level of glutamate to the level of glycine; and
administering cysteine to the individual when the ratio is not within a predetermined range.

2. The method of claim 1, wherein administering the cysteine to the individual establishes homeostasis for cellular absorption.

3. The method of claim 1 wherein measuring the level of glutamate comprises measuring the level of glutamate in a bodily excretion selected from the group consisting of blood and its components; urine; fecal matter; collagen; chyle; interstitial fluid; lymph; extracellular fluid; amniotic fluid; sweat; tears; saliva; mucus; phlegm; hair; fingernails; bone marrow.

4. The method of claim 1, wherein measuring the level of glycine comprises measuring the level of glycine in a bodily excretion selected from the group consisting of blood and its components; urine; fecal matter; collagen; chyle; interstitial fluid; lymph; extracellular fluid; amniotic fluid; sweat; tears; saliva; mucus; phlegm; hair; fingernails; bone marrow.

5. The method of claim 1, wherein measuring the level of glycine and measuring the level of glutamate comprises measuring the level of glycine and glutamate in the same bodily excretion.

6. The method of claim 5, wherein the bodily excretion is blood.

7. The method of claim 1, further comprising:
re-measuring the level of glutamate in the individual;
re-measuring measuring the level of glycine in the individual;
measuring a level of cysteine in the individual; and
determining whether or not homeostasis has been obtained

8. The method of claim 1, wherein the cysteine is administered by provision of orally administered substances, intravenously administered solutions or modification of dietary supplements.

9. A method for establishing homeostasis for neuron firing comprising:
measuring a level of aspartate in an individual;
measuring a level of GABA in the individual;
calculating a ratio of the aspartate level to the glutamate; and
administering GABA, glutamate that is converted to GABA by GAD or GAD to the individual when the ratio is not within a predetermined range.

10. The method of claim 9, wherein administering glutamate or GAD to the individual establishes homeostasis for neuron firing.

11. The method of claim 9, wherein measuring the level of aspartate comprises measuring the level of glutamate in a bodily excretion selected from the group consisting of blood and its components; urine; fecal matter; collagen; chyle; interstitial fluid; lymph; extracellular fluid; amniotic fluid; sweat; tears; saliva; mucus; phlegm; hair; fingernails; bone marrow.

12. The method of claim 9, wherein measuring the level of GABA comprises measuring the level of glycine in a bodily excretion selected from the group consisting of blood and its components; urine; fecal matter; collagen; chyle; interstitial fluid; lymph; extracellular fluid; amniotic fluid; sweat; tears; saliva; mucus; phlegm; hair; fingernails; bone marrow.

13. The method of claim 9, wherein measuring the level of aspartate and measuring the level of GABA comprises measuring the level of aspartate and GABA in the same bodily excretion.

14. The method of claim 13, wherein the bodily excretion is blood.

15. The method of claim 9, further comprising:
re-measuring the level of aspartate in the individual;
re-measuring measuring the level of GABA in the individual;
measuring a level of GAD in the individual; and
determining whether or not homeostasis has been obtained.

16. The method of claim 9, wherein the GAD is administered by provision of orally administered substances, intravenously administered solutions or modification of dietary supplements.

17. A method for obtaining homeostasis in an individual comprising:
measuring a level of a first substance in an individual;
measuring a level of a second substance in an individual;
calculating a ratio of the level of the first substance to the level of the second substance; and
administering a third substance to the individual when the ratio is not within a predetermined range.

18. The method of claim 1, wherein administering the third substance to the individual establishes homeostasis for cellular absorption.

19. The method of claim 1 wherein the first substance is selected from the group consisting of aspartate and glutamate.

20. The method of claim 17, further comprising:
re-measuring the level of the first substance in the individual;
re-measuring measuring the level of the second substance in the individual;
measuring a level of the third substance in the individual; and
determining whether or not homeostasis has been obtained
